(19) United States
(12) Patent Application Publication
(10) Pub. No.: US 2018/0072869 A1
(43) Pub. Date: Mar. 15, 2018

(54) ANTIBACTERIAL POLMER MATERIAL, MANUFACTURING METHOD THEREOF, AND PRODUCT APPLYING THE SAME

(71) Applicant: Tse-Ming Hsin, Westford, MA (US)

(72) Inventor: Tse-Ming Hsin, Westford, MA (US)

(21) Appl. No.: 15/700,200

(22) Filed: Sep. 11, 2017

Related U.S. Application Data
(60) Provisional application No. 62/495,301, filed on Sep. 12, 2016.

(51) Int. Cl. C08K 5/31; C08G 69/26; C08G 63/00; C08K 5/092; C07C 63/26

(52) U.S. Cl. CPC C08K 5/31; C08G 69/265; C07C 63/26; C08K 5/092; C08G 63/00

(57) ABSTRACT

An antibacterial polymer material includes a polymer body and plural antibacterial structures mixed with the polymer body and bonded with the polymer body, and the antibacterial structures have a content of 0.2%-8%. The antibacterial polymer material may be manufactured to form an antibacterial plastic product and an antibacterial fiber, and the antibacterial structures are mixed with the polymer body and bonded with the polymer body, so that the antibacterial ingredient is distributed more uniformly in the product or masterbatch to prevent the antibacterial ingredient from being just distributed on the surface or the antibacterial effect from being lessened or vanished due to a damage of the surface by an external force. Since the antibacterial ingredient is mixed with the masterbatch or product, the possibility of the antibacterial ingredient exuding a corroded or damaged surface is reduced to improve the effect of environmental protection.

### CROSS-REFERENCE TO RELATED APPLICATIONS

This non-provisional application claims priority under 35 U.S.C. § 119(e) on Patent Application No(s). 62/495,301 filed in the United States on Sep. 12, 2016, the entire contents of which are hereby incorporated by reference.

### FIELD OF INVENTION

The present invention relates to the field of antibacterial polymer materials, in particular to an antibacterial polymer material with an antibacterial ingredient which does not exude and provides a longer antibacterial effect, as well as its manufacturing method, and a product applying the antibacterial polymer material and manufacturing method.

### BACKGROUND OF INVENTION

### Description of the Related Art

In recent years, major disease disasters frequently occur; thus, public health and personal hygiene come particularly important. In various ways of improving health and hygiene, more and more people began to pay attention to the use of antibacterial products for disease prevention. However, natural or synthetic products generally do not have an antibacterial function, and such products without the antibacterial function become parasitic by microorganisms easily, particularly in warm and humid places.

To provide the antibacterial function of a product, some manufacturers have attempted applying various types of posttreatments to the surface of products. Although such posttreatment method can improve the antibacterial effect quickly, the method still has the following drawbacks. After posttreatment, the antibacterial function of a product may disappear after a few times of use. In addition, the ingredient with the antibacterial function may be damaged by external factors such as liquid erosion or surface damage, so that the antibacterial ingredient on the surface may be exuded or discharged to contaminate the environment. In addition, posttreatments typically involve very complicated manufacturing processes and incurs a high cost, and thus the use of such product is not popular.

As to the issue of the antibacterial function disappearing quickly after a few times of use, some manufacturers have introduced a new method to fix the antibacterial ingredient onto the surface of the product as disclosed in U.S. Pat. Nos. 5,817,325, 6,030,632, 6,126,931, 6,264,936 and 7,288,264, wherein the antibacterial ingredient forms a crosslink network through a crosslinking agent such as derivatives of epoxide or isocyanate in order to fix the antibacterial ingredient to the surface of the product. Similar to conventional posttreatments, the aforementioned technologies just can distribute the antibacterial ingredient on the surface of product only, but no antibacterial ingredient is distributed in the product such as the position under the surface of the product. If the surface of the product is pulled, stretched, worn, eroded or damaged, the antibacterial effect will be lost since the distributed position of the antibacterial ingredient is ruined. In addition, the other two aforementioned drawbacks are still not overcome yet.

In view of the drawbacks of the prior art, the inventor of the present invention based on years of experience in the related industry to conduct extensive research and development, and finally developed and provided an antibacterial polymer material, a manufacturing method thereof, and a product applying the same, in hope of overcoming the aforementioned drawbacks of the prior art.

### Summary of the Invention

In view of the aforementioned drawbacks of the prior art, it is a primary objective of the present invention to provide an antibacterial polymer material, a manufacturing method thereof, and a product applying the same, and the invention not only provides the antibacterial activity to the surface and the entire body of products, but also provides the antibacterial material with the non-leaching feature and environmentally friendly.

To achieve the aforementioned and other objectives, the present invention provides an antibacterial polymer material, having a polymer body and a plurality of antibacterial structures, characterized in that the antibacterial structures are mixed with the polymer body and bonded with the polymer body, and the content of antibacterial structures is 0.2%-8%, and the antibacterial polymers have a general formula of:

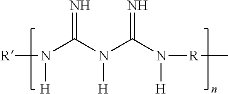

wherein, R′ represents nylon or polyester; R represents an alkyl group, alkenyl group, alkynyl group, alicyclic group, or aryl group of a substituted carbon atom or non-substituted carbon; and n is an integer greater than or equal to 1, so that a longer antibacterial effect is provided, and the antibacterial effect of the antibacterial polymer material is not affected by a damaged surface.

Wherein, the antibacterial polymers preferably have a chemical structure of

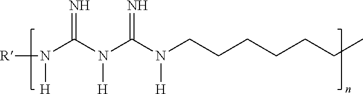

and R′ represents nylon or polyester; n is an integer greater than or equal to 1 to smaller than or equal to 40, to achieve a better antibacterial quality.

To achieve the aforementioned and other objectives, the present invention further provides a manufacturing method of an antibacterial polymer material, comprising the steps of: mixing a diamine and a dicarboxylic acid with each other to form a polymer body, and the ratio of the diamine to the dicarboxylic acid being equal to 0.5-1.5; mixing the polymer body and the plurality of antibacterial structures with each other in nitrogen gas and an environmental condition at a temperature of 200° C.-220° C. for 1-4 hours to form a mixture, wherein the content ratio of the antibacterial structures in the mixture is 0.2%-8%; and polymerizing the mixture under the conditions at a temperature of 260° C.-300° C. and a pressure of 300 torr-3 torr for 4-8 hours to form an antibacterial polymer material, so that the antibacterial structures are fully distributed in the antibacterial polymer material.

Wherein, the diamine is hexamethylene diamine, butanediamine, nonanediamine, decanediamine, dodecane diamine, m-xylylenediamine or their derivatives, and different antibacterial polymer materials may be manufactured according to user requirements.

In addition, the dicarboxylic acid is adipic acid, sebacic acid, dodecanedioic acid, terephthalic acid, isophthalic acid or their derivatives, and different antibacterial polymer materials may be manufactured according to user requirements.

Preferably, the antibacterial polymer have a chemical structure of:

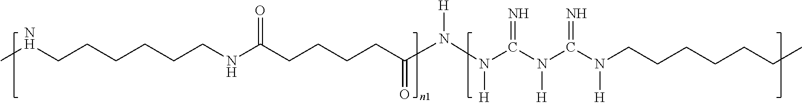

wherein, n1 is any integer greater than 1 and this represents the structure of nylon 66; n is an integer greater than or equal to 1 to smaller than or equal to 40.

To achieve the aforementioned and other objectives, the present invention further provides a manufacturing method of another antibacterial polymer material, comprising:

mixing a diol and a dicarboxylic acid with each other to form a polymer body, and the ratio of the diol to the dicarboxylic acid being 0.5-1.5;

mixing the polymer body and the plurality of antibacterial structures with each other in nitrogen gas in an environmental condition at a temperature of 200° C.-220° C. for 2 hours to form a mixture, wherein the mixture has a content ratio of 0.2%-8% of the antibacterial structures;

polymerizing the mixture under the conditions at a temperature of 250° C.-300° C. and an air pressure of 100 torr-1 torr for 6-8 hours to form an antibacterial polymer material, so that the antibacterial structure is fully distributed in the antibacterial polymer material.

Wherein, the diol is ethylene glycol, 1,4-butanediol, 1,3-propylene glycol or their derivatives, and different antibacterial polymer materials may be manufactured according to user requirements.

In addition, the dicarboxylic acid is adipic acid, sebacic acid, dodecanedioic acid, terephthalic acid, isophthalic acid or their derivatives, and different antibacterial polymer materials may be manufactured according to user requirements.

Preferably, the antibacterial structures have a chemical structure of:

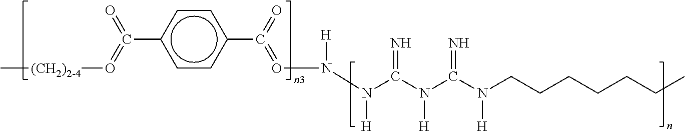

n is an integer greater than or equal to 1 to smaller than or equal to 40.

To achieve the aforementioned and other objectives, the present invention further provides an antibacterial plastic product made of a plurality of antibacterial polymer materials and a plastic material mixed with each other, characterized in that each of the antibacterial polymer materials has a polymer body and a plurality of antibacterial structures, and the antibacterial structures are mixed with the polymer body and bonded with the polymer body, and the antibacterial polymer materials have a content of 0.2%-8% of the antibacterial structures, and the antibacterial structures have a general formula of:

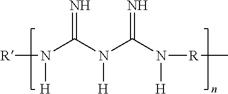

wherein, R′ represents nylon or polyester; R represents an alkyl group, alkenyl group, alkynyl group, alicyclic group, or aryl group of a substituted carbon atom or non-substituted carbon atom, and n is an integer greater than or equal to 1; and a content of the antibacterial structures in the antibacterial plastic product is 0.002%-0.8%, preferably 0.02%-0.4%, so that a longer antibacterial effect is provided, and the antibacterial effect of the antibacterial polymer material is not affected by a damaged surface.

To achieve the aforementioned or other objectives, the present invention further provides an antibacterial fiber,

formed by mixing a plurality of antibacterial polymer materials and a plurality of fiber materials, and then spinning the mixture to form the antibacterial fibers, characterized in that each of the antibacterial fibers has a polymer body and a plurality of antibacterial structures, and the antibacterial structures are mixed with the fiber polymer body and bonded with the fiber polymer body, and the antibacterial structures in the antibacterial fibers have a content of 0.2%-8%, and the antibacterial structures have a general formula of:

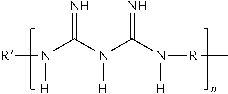

wherein, R′ represents nylon or polyester; R represents an alkyl group, alkenyl group, alkynyl group, alicyclic group, aryl group of a substituted carbon atom or non-substituted carbon atom, and n is an integer greater than or equal to 1; and the antibacterial fiber have a content of 0.002%-0.8% of the antibacterial structures, and preferably 0.02%-0.4%, so that a longer antibacterial effect is provided, and the antibacterial effect of the antibacterial polymer material is not affected by a damaged surface.

Wherein, the fiber material is an artificial fiber polymer to facilitate mixing the fiber material with the antibacterial polymer material and spinning the mixture.

In summation of the description above, the antibacterial structures are mixed with the polymer body and bonded with the polymer body, so that the antibacterial ingredient is distributed more uniformly in the product or masterbatch to prevent the antibacterial ingredient from being just distributed on the surface or the antibacterial effect from being lessened or vanished due to a damage of the surface by an external force. Since the antibacterial ingredient is mixed with the masterbatch or product, the possibility of the antibacterial ingredient exuding a corroded or damaged surface is reduced to improve the effect of environmental protection.

### BRIEF DESCRIPTION OF THE DRAWINGS

FIG. 1 is a cross-sectional view of an antibacterial polymer material of the present invention;

FIG. 2 is a first flow chart of a manufacturing process of an antibacterial polymer material in accordance with the present invention;

FIG. 3 is a second flow chart of a manufacturing process of an antibacterial polymer material in accordance with the present invention;

FIG. 4 is a cross-sectional view of an antibacterial plastic product in accordance with the present invention; and

FIG. 5 is a cross-sectional view of an antibacterial fiber in accordance with the present invention.

### DESCRIPTION OF THE PREFERRED EMBODIMENTS

The above and other objects, features and advantages of this disclosure will become apparent from the following detailed description taken with the accompanying drawings.

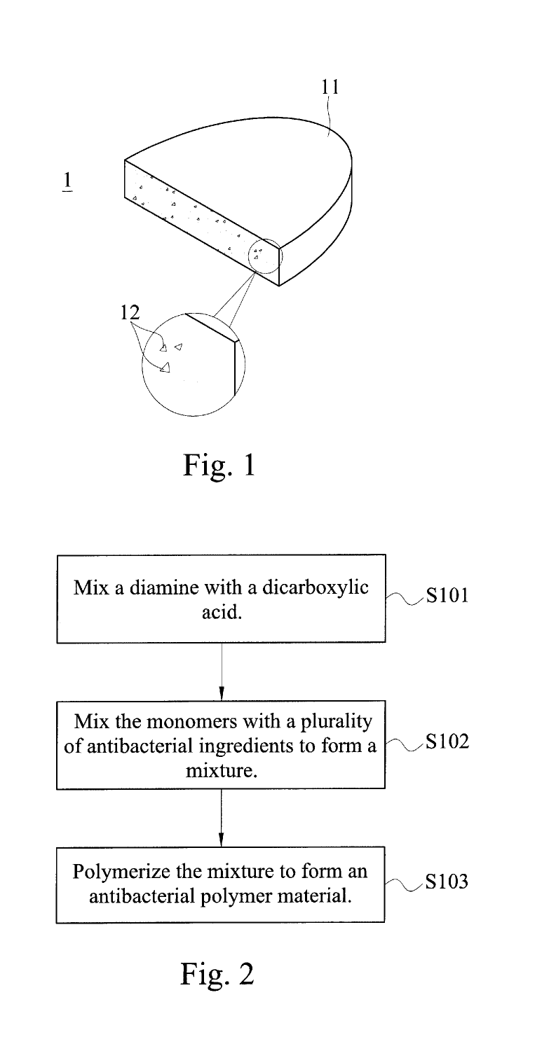

With reference to FIG. 1 for a cross-sectional view of an antibacterial polymer material of the present invention, the antibacterial polymer material 1 comprises a polymer body 11 and a plurality of antibacterial structures 12, characterized in that the antibacterial structures 12 are mixed with the polymer body 11 and bonded with the polymer body 11, and the content of antibacterial structures 12 is 0.2%-8%, and the antibacterial polymer 12 has a general formula of:

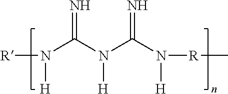

wherein, R′ represents nylon or polyester; R represents an alkyl group, alkenyl group, alkynyl group, alicyclic group, or aryl group of a substituted carbon atom or non-substituted carbon atom, and n is an integer greater than or equal to 1.

In this embodiment, the antibacterial polymer 12 have a chemical structure of:

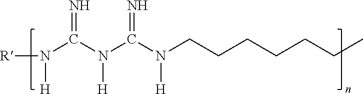

Wherein, R′ represents nylon or polyester; n is an integer greater than or equal to 1 or smaller than of equal to 40. Such chemical structure provides a better antibacterial effect.

In addition, the antibacterial structures 12 may have a chemical structure of:

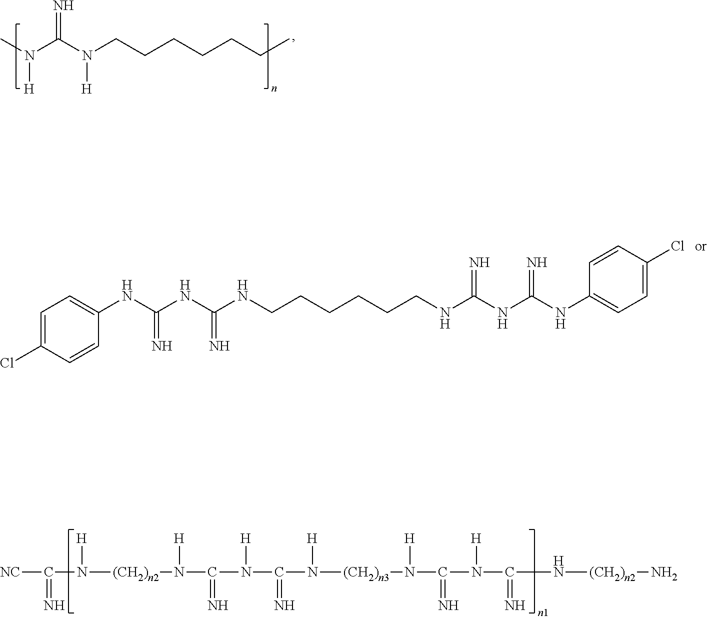

Wherein, n and n1 are integers greater than or equal to 1, and n2 and n3 are one selected from the integers of 2, 4, and 6. For example, n2=2 and n3=6.

The method of mixing the antibacterial structures 12 into the polymer body 11 can improve the persistence of the antibacterial ingredient. In addition, since the antibacterial ingredient is not just distributed on the surface, therefore it is not necessary to use the crosslinking agent, so as to lower the cost of the manufacturing process and simplify the manufacturing process. Since the antibacterial ingredient is not just distributed on the surface only, therefore if the surface is damaged, other parts of the antibacterial polymer material 1 still have the antibacterial ingredient, and the antibacterial effect will not be affected too much. In addition, if the content of the antibacterial structures 12 is less than 0.002%, the antibacterial effect of the antibacterial polymer material 1 will be affected significantly. Since the antibacterial polymer materials may be used for manufacturing various plastic products or fibers, therefore the antibacterial polymer materials may be contacted with the user. If the content of the antibacterial structures 12 exceeds 8%, the antibacterial effect will keep increasing, but it may cause skin irritation, so that the content of 8% is preferred to maintain a balance between the antibacterial effect and the stimulation.

With reference to FIG. 2 for a first flow chart of a manufacturing process of an antibacterial polymer material in accordance with the present invention, the manufacturing process of the antibacterial polymer material 1 comprises the following steps:

S101: Mix a diamine and a dicarboxylic acid with each other to form a polymer body 11, and the ratio of the diamine to the dicarboxylic acid is 0.5-1.5, and the diamine may be hexamethylene diamine, butanediamine, nonanediamine, decanediamine, dodecane diamine, m-xylylenediamine, or their derivatives, and the dicarboxylic acid may be adipic acid, sebacic acid, dodecanedioic acid, terephthalic acid, isophthalic acid or their derivatives, wherein the diamine includes 600 g of hexamethylene diamine, and the dicarboxylic acid includes 754 g of adipic acid.

S102: Mix the polymer body 11 and 50 g of the solution containing the antibacterial structures 12 with a concentration of 20%. During the mixing process, a high-pressure reaction chamber using nitrogen gas as the background gas is provided for mixing the polymer body 11 with the antibacterial structures 12 at a temperature of 200° C.-220° C. for 2 hours to form a mixture, wherein the antibacterial structures 12 has a content of approximately 0.7% (which falls between 0.2% and 8%), and the temperature is 200° C.

S103: Drop the pressure of the high-pressure reaction chamber to one atmosphere and maintain this pressure for an hour, and then increase the temperature slowly to 250° C.-300° C., and then vacuum (with an air pressure approximately 300 torr-3 torr) for 4-8 hours to perform a polymerization of the mixture, and finally manufacture the antibacterial polymer material 1. Wherein, the temperature is 260-280° C., and the vacuum time is approximately 5-6 hours.

With such manufacturing process, the antibacterial structures 12 are distributed in the polymer body 11 to provide a relatively longer antibacterial effect and prevent the antibacterial effect of the antibacterial polymer material 1 from being disappeared due to the damaged surface. In this manufacturing process, the structure after the combination of the polymer body and the antibacterial structures is given below:

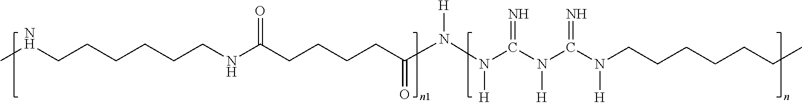

In another embodiment, the manufacturing process of the antibacterial polymer material 1 may use other monomers to form polymer body 11. Now, the monomer is a cyclic amide compound such as caprolactam (which is 6-aminocaproic acid after hydrolysis). Alternatively, monomers can be used to form polymer body 11 are 11-aminoundecanoic acid (with a chemical formula: HOOC(CH₂)₁₀(NH₂), 12-aminododecanoic acid (with a chemical formula: HOOC(CH₂)₁₁(NH₂) or their derivatives. Then, the polymer body 11 and the antibacterial structures 12 are mixed with each other in nitrogen gas and an environmental condition at a temperature of 180-240° C. for 1-4 hours to form a mixture, wherein the antibacterial structures 12 in the mixture have a content ratio of 0.2%-8%. Then, the mixture is polymerized under the conditions of a temperature of 240° C.-300° C. and an air pressure of 300 torr-3 torr for 4-8 hours to form an antibacterial polymer material 1, and the antibacterial structure 12 is fully distributed in the antibacterial polymer material 1. In this manufacturing process, the polymer body 11 and the antibacterial structures 12 are combined to have a structural formula of:

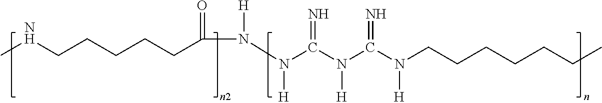

In addition, the cyclic amide compounds and its derivative of another embodiment may form a composite antibacterial nylon material such as Nylon6/66 prepared by polymerizing the mixed monomers with the antibacterial ingredient.

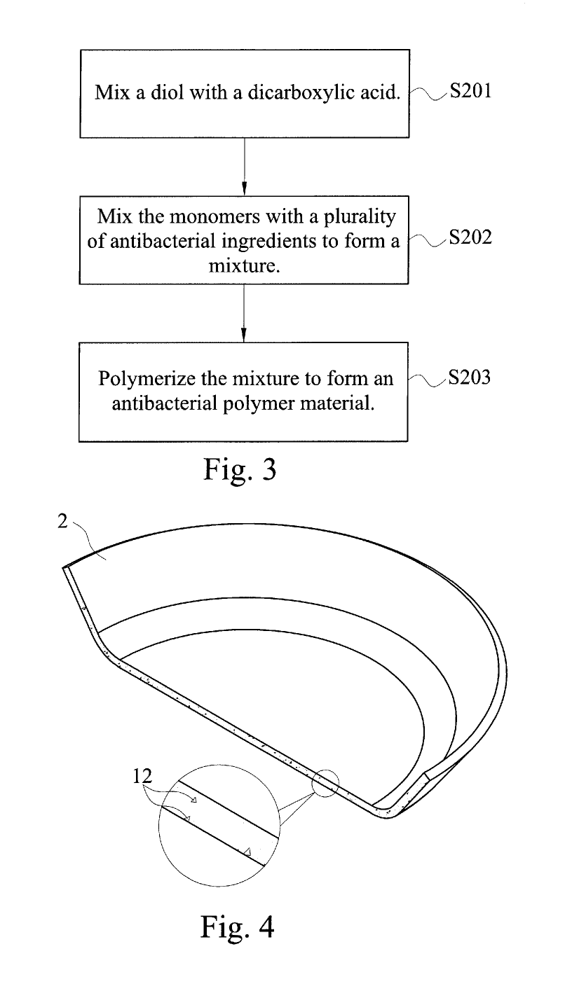

With reference to FIG. 3 for a second flow chart of a manufacturing process of another antibacterial polymer material in accordance with the present invention, the method is provided for manufacturing another antibacterial polymer material 1 by different materials, which are used to manufacture an antibacterial product according to different requirements.

S201: Mix a diol and a dicarboxylic acid with each other to form a polymer body 11, wherein the ratio of the diol to the dicarboxylic acid is 0.5-1.5, and the diol is ethylene glycol, 1,4-butanediol, 1,3-propylene glycol or their derivatives, and the dicarboxylic acid is adipic acid, sebacic acid, dodecanedioic acid, terephthalic acid, isophthalic acid or their derivatives, and the diol includes 330 g of 1,4-butanediol, and the dicarboxylic acid includes 600 g of terephthalic acid.

S202: Mix the polymer body 11 with 50 g of a solution containing 20% of the antibacterial structures. During the mixing process, a high-pressure reaction chamber using nitrogen gas as the background gas is provided for mixing the polymer body 11 with the antibacterial structures 12 in the condition at a temperature of 200° C.-220° C. for 2 hours to form a mixture. Wherein, the antibacterial structures 12 have a content of approximately 1.1%, which falls within a range between 0.2% and 8%, and the temperature is 200° C.

S203: Increase the temperature to 250° C.-300° C. and vacuum the mixture (at an air pressure of 100 torr-1 torr) for 6-8 hours to remove the diol and water from the mixture for polymerization, and finally manufacture the antibacterial polymer material 1, wherein the temperature is 260° C., and the vacuum time is 7 hours.

In an alternative manufacturing method, 20 g of the antibacterial structures 12 and 1000 g of the manufactured polymer body 11 are mixed with each other, and the aforementioned manufacturing process is used to manufacture the antibacterial structures 12 with a content of approximately 2% of the antibacterial polymer material 1. In these manufacturing processes, the polymer body and the antibacterial structures are combined to have the structural formula given below:

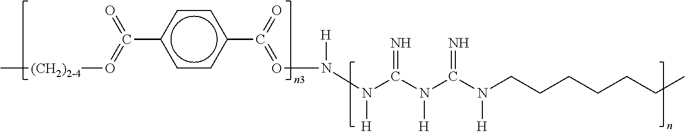

With the aforementioned manufacturing process, the antibacterial structures 12 are distributed in the polymer body 11 to produce the antibacterial polymer material 1 with a persistent antibacterial effect and prevent the antibacterial effect from disappearing due to the damaged surface, and the antibacterial polymer material 1 of different materials can be manufactured according to different requirements.

With reference to FIG. 4 for a cross-sectional view of an antibacterial plastic product of the present invention, the antibacterial plastic product 2 is manufactured by mixing the antibacterial polymer materials 1 with a plastic material, and the content of antibacterial structures 12 in the antibacterial plastic product 2 is 0.002%-0.8%, preferably 0.02%-0.4%. During the use of antibacterial plastic product 2, if the content of the antibacterial ingredient is too low, there will be no antibacterial effect. If the content of the antibacterial ingredient is too high, the user's skin will be stimulated. Therefore, the content of the antibacterial ingredient is set to 0.002%-0.8% and preferably 0.02%-0.4% to obtain the best antibacterial effect. The remaining parts of the technical characteristics of the antibacterial polymer material 1 have been described in the previous embodiment as shown in FIG. 1, and thus will not be repeated.

The following table lists the data related to the antibacterial effect of the manufactured antibacterial plastic product 2 of the present invention. ASTM E2149-13a is used for inspecting the antibacterial plastic product 2, and this method is applicable for testing the antibacterial materials which are used in normal conditions and will not be dispersed to the environment naturally. To ensure a good contact between bacteria and the synthetic material, the material with a larger size requires an inspection time of 24 hours. In the process, a sample is put on a vibrator with the maximum stroke. The bacteria to be tested are staphylococcus aureus (ATCC 6538) and Klebsiella pneumoniae (ATCC 4352).

TABLE 1

| | | | Bacteria | |
|---|---|---|---|---|
| | | | Decrease | Contact |
| Sample | Bacteria | Method | Level (%) | Time (h) |
| Embodiment 1 | Staphylococcus | ASTM | >99.99 | 24 |
| | aureus | E2149-13a | | |
| | Klebsiella | ASTM | 99.98 | 24 |
| | pneumoniae | E2149-13a | | |
| Embodiment 2 | Staphylococcus | ASTM | 99.24 | 24 |
| | aureus | E2149-13a | | |
| | Klebsiella | ASTM | 99.93 | 24 |
| | pneumoniae | E2149-13a | | |

The antibacterial plastic product 2 of Embodiment 1 is formed by combining the antibacterial structures 12 with the plastic material of a nylon material, and the antibacterial plastic product 2 of Embodiment 2 is formed by combining the antibacterial structures 12 with the plastic material of a polyester material. The aforementioned data show that the antibacterial plastic product 2 has a very significant antibacterial effect. Since the antibacterial structures 12 are distributed in the antibacterial plastic product 2, therefore the antibacterial effect is not affected too much by the damaged surface.

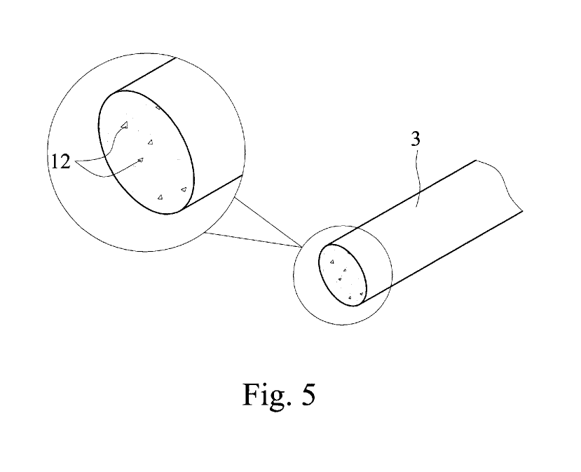

With reference to FIG. 5 for a cross-sectional view of an antibacterial fiber of the present invention, the antibacterial fiber 3 is formed by mixing the aforementioned antibacterial polymer materials 1 and fiber materials with each other and spinning the mixture, wherein a content of the antibacterial structures 12 in the antibacterial fiber 3 is 0.002%-0.8%, preferably 0.02%-0.4%. In the use of the antibacterial fiber 3, if the content of the antibacterial ingredient is too low, there will be no antibacterial effect. If the content of the antibacterial ingredient is too high, the stimulation to the user's skin will occur. Therefore, the content of the antibacterial ingredient is set to 0.002%-0.8%, preferably 0.02%-0.4% to obtain the best antibacterial effect for the reasons as described above.

The following table lists the data related to the antibacterial effect of the manufactured textile by using the antibacterial fiber 3 of the present invention, wherein the fiber materials of the textile are artificial fibers. The AATCC 100 method is used for inspecting the antibacterial fiber 3, and the bacteria to be inspected are klebsiella pneumoniae (ATCC 4352), chaetomium globosum (ATCC 6205), staphylococcus aureus (ATCC 6538) and escherichia coli (ATCC 8739). In the method, the textile is cut into a round sample with a diameter of 4.8±0.1 cm, and sufficient samples are accumulated to absorb 1.0±0.1 ml of the inoculum, and the sample is put into a 250 mL wide-mouth glass jar with a screw cap, and the sample contains 1.0±0.1 ml of bacterial culture (wherein the inoculum contains 1-2×10⁵ cfu/mL), and the glass jar is sealed for the nurture for 24 hours. After the nurture with a contact time of 24 hours, the 100+1 ml of the neutral solution is added into the jar, and then the jar is shaken violently for 1 minute. To ensure the concentration of the surviving bacteria, a continuous dilution method is performed on the nutrient agar plate. For the antibacterial test, incubation is taken place on the gar plate at 35±2° C. for 48±2 hours.

TABLE 2

| | | Bacteria Decrease | |
|---|---|---|---|
| Bacteria | Method | Level (%) | Contact Time (h) |
| Klebsiella | AATCC 100 | >99.99 | 24 |
| pneumoniae | | | |
| Chaetomium | AATCC 100 | 96.38 | 24 |
| globosum | | | |
| Staphylococcus | AATCC 100 | >99.99 | 24 |
| aureus | | | |
| Escherichia coli | AATCC 100 | >99.99 | 24 |

The aforementioned data show that the textile manufactured by the antibacterial fiber 3 has a very significant antibacterial effect. Since the antibacterial structures 12 are distributed in the antibacterial fiber 3, therefore the antibacterial effect is not affected too much by the damaged textile surface.

In an embodiment, the antibacterial polymer materials 1 may be mixed with other plastic materials to form a mixture, and the antibacterial polymer materials 1 in the mixture have a content of 4% by weight, and then the mixture is used to manufacture nonwoven fabric. The following table lists the data related to the antibacterial effect data of the nonwoven fabric having the antibacterial structures 12. ASTM 2149-13a is used for inspecting the unwoven fabric, and the bacterium to be tested is staphylococcus aureus (ATCC 6538).

TABLE 3

| | | Bacteria Decrease | |
|---|---|---|---|
| Bacteria | Method | Level (%) | Contact Time (h) |
| Staphylococcus | ASTM 2149-13a | 97.58 | 24 |
| aureus | | | |

The aforementioned data show that the nonwoven fabric with the antibacterial structures 12 has a very significant antibacterial effect. Since the antibacterial structures 12 are distributed in the nonwoven fabric, therefore the antibacterial effect is not affected too much by the damaged surface.

In the test for testing the persistence of the antibacterial effect of the present invention, the textile made of the antibacterial fiber 3 is washed by water at room temperature for 30 times, and then staphylococcus aureus is inoculated to the textile with contact time of 24 hours, and the bacteria reduction is still greater than 99.99%. Compared with the data in Table 2, we clearly see that the antibacterial effect of the textile is the same before and after 30 washing cycles. Therefore, the present invention surely has a persistent antibacterial effect.

In the test for examining whether or not the antibacterial ingredient is exuded or discharged, 0.3 g of the antibacterial plastic fiber is mixed with 30 ml of de-ionized water, and the mixture is shaken at room temperature for 24 hours, and then filtered by water solution, and chromatography is used for measuring the concentration of the antibacterial structures 12 in the aqueous solution, and the result is shown in the table below.

TABLE 4

| | Concentration of antibacterial |
|---|---|
| Sample | structure in solution |
| Antibacterial plastic product | Not detected |

Since the threshold value of the inspection is 1 ppm. In other words, the concentration must be greater than 1 ppm before it can be detected. Obviously, the antibacterial structure 12 of the present invention is almost not exuded or discharged at all, and there is no issue of affecting the environment or causing the pollution.

In summation of the description above, the present invention using the method of distributing the antibacterial structures 12 in the antibacterial polymer material 1 to provide a more persistent antibacterial effect for the manufactured antibacterial plastic product 2 and antibacterial fiber 3, and the antibacterial structures 12 are not just disposed on the surface only, so that the antibacterial effect will not be lost due to the damaged surface. In addition, the antibacterial structures 12 are not exuded or discharged easily to achieve the environmental protection effect. Compared with the prior art, the invention has the advantages of a simpler manufacturing process that requires no additional post treatment such as adding a crosslinking agent, so as to lower the manufacturing cost and improve the manufacturing efficiency.

What is claimed is:

1. An antibacterial polymer material, having a polymer body and a plurality of antibacterial structures, characterized in that the antibacterial structures are mixed with the polymer body and bonded with the polymer body, and a content of the antibacterial structures is 0.2%-8%, and the antibacterial structures have a general formula of 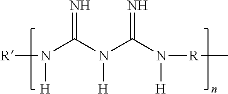
wherein, R′ represents nylon or polyester; R represents an alkyl group, alkenyl group, alkynyl group, alicyclic group, or aryl group of a substituted carbon atom or non-substituted carbon; and
n is an integer greater than or equal to 1.

2. The antibacterial polymer material of claim 1, wherein the antibacterial structures have a chemical structure of 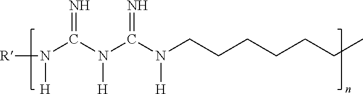
wherein, R′ represents nylon or polyester; n is an integer greater than or equal to 1 to smaller than or equal to 40.

3. A manufacturing method of an antibacterial polymer material, comprising the steps of:
mixing a diamine with a dicarboxylic acid to form a polymer body, and the ratio of the diamine to the dicarboxylic acid being equal to 0.5-1.5;
mixing the polymer body with the plurality of antibacterial structures in nitrogen gas and an environmental condition at a temperature of 200° C.-220° C. for 1-4 hours to form a mixture, wherein the mixture has a content ratio of 0.2%-8% of the antibacterial structure; and
polymerizing the mixture under the conditions at a temperature of 260° C.-300° C. and an air pressure of 300 torr-3 torr for 4-8 hours to form an antibacterial polymer material.

4. The method of claim 3, wherein the diamine is one selected from the group consisting of hexamethylene diamine, butanediamine, nonanediamine, decanediamine, dodecane diamine, m-xylylenediamine, and a derivative thereof.

5. The method of claim 3, wherein the dicarboxylic acid is one selected from the group consisting of adipic acid, sebacic acid, dodecanedioic acid, terephthalic acid, isophthalic acid, and a derivative thereof.

6. The method of claim 3, wherein the antibacterial structures have a chemical structure of 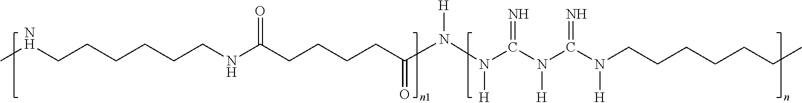
wherein, n and n1 are integers greater than or equal to 1.

7. A manufacturing method of an antibacterial polymer material, comprising the steps of:
mixing a diol with a dicarboxylic acid to form a polymer body, and the ratio of the diol to the dicarboxylic acid being 0.5-1.5;
mixing the polymer body with the plurality of antibacterial structures in nitrogen gas in an environmental condition at a temperature of 200° C.-220° C. for 2 hours to form a mixture, wherein the mixture has a content ratio of 0.2%-8% of the antibacterial structures;
polymerizing the mixture under the conditions at a temperature of 250° C.-300° C. and an air pressure of 100 torr-1 torr for 6-8 hours to form an antibacterial polymer material.

8. The method of claim 7, wherein the diol is one selected from the group consisting of ethylene glycol, 1,4-butanediol, 1,3-propylene glycol, and a derivative thereof.

9. The method of claim 7, wherein the dicarboxylic acid is one selected from the group consisting of adipic acid, sebacic acid, dodecanedioic acid, terephthalic acid, isophthalic acid and a derivative thereof.

10. The method of claim 7, wherein the antibacterial structures have a chemical structure of 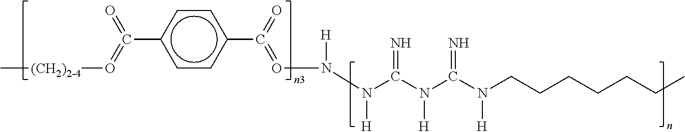
wherein, n and n3 are integers greater than or equal to 1.

11. An antibacterial plastic product, made of a plurality of antibacterial polymer materials and a plastic material mixed with each other, characterized in that each of the antibacterial polymer materials has a polymer body and a plurality of antibacterial structures, and the antibacterial structures are mixed with the polymer body and bonded with the polymer body, and the antibacterial polymer materials contain a content of 0.2%-8% of the antibacterial structures, and the antibacterial structures have a general formula of 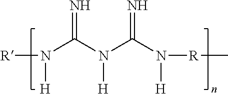
wherein, R′ represents nylon or polyester; R represents an alkyl group, alkenyl group, alkynyl group, alicyclic group, or aryl group of a substituted carbon atom or non-substituted carbon atom, and n is an integer greater than or equal to 1; and the antibacterial plastic product contain a content of 0.002%-0.8% of the antibacterial structures.

12. The antibacterial plastic product of claim 11, wherein a content of the antibacterial structures in the antibacterial plastic product is 0.02%-0.4%.

13. An antibacterial fiber, formed by mixing a plurality of antibacterial polymer materials and a plurality of fiber materials mixed with each other, and then spinning the mixture to form the antibacterial fibers, characterized in that each of the antibacterial polymer materials has a polymer body and a plurality of antibacterial structures, and the antibacterial structures are mixed with the polymer body and bonded with the polymer body, and the antibacterial polymer materials contain a content of 0.2%-8% of the antibacterial structures, and the antibacterial structures have a general formula of 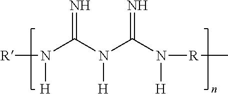
wherein, R′ represents nylon or polyester; R represents an alkyl group, alkenyl group, alkynyl group, alicyclic group, aryl group of a substituted carbon atom or non-substituted carbon atom, and n is an integer greater than or equal to 1; and the antibacterial fiber contains a content of 0.002%-0.8% of the antibacterial structures.

14. The antibacterial fiber of claim 13, wherein a content of the antibacterial structures in the antibacterial fiber is 0.02%-0.4%.

15. The antibacterial fiber of claim 13, wherein the fiber materials are artificial fibers.
